(19) United States
(12) Patent Application Publication
(10) Pub. No.: US 2025/0377298 A1
(43) Pub. Date: Dec. 11, 2025

(54) WATER QUALITY SENSOR

(71) Applicant: SOLTEAM OPTO, INC., Taoyuan (TW)

(72) Inventors: Chih-Teng CHENG, Taoyuan (TW); Chien-Kuo WANG, Taoyuan (TW); Chun-Yen WU, Taoyuan (TW); Chia-Hao CHANG, Taoyuan (TW); Chin-Feng CHEN, Taoyuan (TW)

(21) Appl. No.: 19/098,079

(22) Filed: Apr. 2, 2025

(30) Foreign Application Priority Data
Jun. 6, 2024 (TW) 113121055

(51) Int. Cl. G01N 21/53; D06F 34/22; G01N 21/94; G01N 33/18

(52) U.S. Cl. CPC G01N 21/53; D06F 34/22; G01N 21/94; G01N 33/18

(57) ABSTRACT

A water quality sensor includes a housing that defining a channel between a first light-transmission portion and a second light-transmission portion thereof, and a sensing module including a circuit board mounted in the housing, a light emitting element provided on a first surface of the circuit board and disposed in the first light-transmission portion, a first light receiving element provided on the first surface of the circuit board and disposed in the second light-transmission portion and a second light receiving element provided on an opposing second surface of the circuit board. The light emitting element transmits light signal to the first light receiving element through the channel to form a first light path. The second light receiving element receives scattered light of impurity particles in the water flow irradiated by the light signal on the first light path and forms a second light path.

This application claims the priority benefit of Taiwan patent application number 113121055, filed on Jun. 6, 2024.

### BACKGROUND OF THE INVENTION

### 1. Field of the Invention

The present invention relates to water quality sensor technology, in particular to a water quality sensor, wherein the light emitting element and the first light receiving element belong to a light transmission structure, so that when washing water flows through a first light path, the output voltage difference of the first light receiving element is inversely proportional to the turbidity; the light emitting element and the second light receiving element belong to a scattered light structure and share the same light emitting element with the first light receiving element, so that the output voltage difference of the second light receiving element is proportional to the turbidity. The special thing is that the second light receiving element for receiving the scattered light and the light emitting element are respectively located on two opposite surfaces of the circuit board. The output voltage of the first light receiving element is read by the microcontroller unit of the washing appliance, which determines whether the washing appliance uses the first light receiving element or the second light receiving element to calculate the water turbidity and washing time. The water quality sensor can accurately judge and avoid the subsequent problems caused by the washing appliance using the wrong wash cycle intensity (wasting washing water and electricity or failing to clean the items).

### 2. Description of the Related Art

Currently, smart washing appliances (dishwashers, washing machines) all use turbidity sensors to determine the wash cycle (low, medium, high) based on the turbidity of the washing water. If the turbidity is high, the medium-high wash cycle is used, and if the turbidity is low, the low wash cycle is used, so as to achieve intelligent energy-saving function (saving water and electricity). Please refer to FIG. 1, which is a schematic diagram of the structure of a prior art water quality sensor. Due to cost considerations, most turbidity sensors A1 currently available on the market use a set of light-transmitting and light-receiving elements (including: light transmitter A2 and light receiver A3), and direct transmission is used between the light transmitter A2 and the light receiver A3 for detection. From a curve graph showing the ratio of turbidity to output voltage of a conventional light receiving element in FIG. 2 and a ratio bar graph of the conventional light receiving element at different turbidities and output voltage differences in FIG. 3, it can be seen that the higher the turbidity, the smaller the signal voltage difference. The transmissive structure has a higher resolution (larger voltage difference) at low turbidity of 40˜1000 NTU; a lower resolution at high turbidity of 2000˜4000 NTU; and there is almost no resolution at ultra-high turbidity above 4000 NTU.

However, the voltage difference in the turbidity ranges of 2000˜4000 NTU is only about 0.6V. Considering that water will flow during the detection of the washing appliance, the voltage signal error will become larger (about 0.1˜0.3V floating), so the resolution in the turbidity ranges of 2000˜4000 is reduced, which makes it easy to misjudge.

Furthermore, when using the turbidity sensor A1, when pollutants are prepared according to the standard, only the pollutant ratio of 50% (about 1400±300 NTU) can be distinguished. It is difficult to distinguish between the two when the pollutant ratio is 100% (2600±300 NTU) and 150% (3300±300 NTU), and it is easy to judge the wrong wash cycle in actual application. For example, if the pollutant ratio is 100%, the medium wash cycle should be used for washing, and if the pollutant ratio is 150%, the high wash cycle should be used for washing. However, there is a high probability that the turbidity sensor A1 will misjudge the pollutant ratio as 100% as a high wash cycle, resulting in a waste of washing water and electricity. Another situation is that the turbidity sensor A1 misjudged the pollutant ratio of 150% as a medium wash cycle, resulting in using too little washing water and electricity for washing so that the items cannot be washed clean. The various problems caused by the aforementioned turbidity sensor A1 due to inaccurate judgment need to be studied and solved by those in the industry.

### SUMMARY OF THE INVENTION

Therefore, in view of the above problems and deficiencies, the inventor collected relevant information and, after multiple evaluations and considerations, designed the invention of this water quality sensor.

The main object of the present invention is to provide a water quality sensor, which comprises a housing and a sensing module. The housing comprises a holder base and a cover which can be connected to each other. The holder base comprises a holder base body, a first light-transmission portion and a second light-transmission portion protruded from one side of the holder base body, a channel formed between the first light-transmission portion and the second light-transmission portion for water to flow through, and a receiving chamber defined in the holder base body in communication with the first light-transmission portion and the second light-transmission portion. The sensing module comprises a circuit board installed in the receiving chamber and extended into the first light-transmission portion and the second light-transmission portion, a light emitting element provided on a first surface of the circuit board and disposed in the first light-transmission portion, at least one first light receiving element provided on the first surface of the circuit board and disposed in the second light-transmission portion, and at least one second light receiving element provided on an opposing second surface of the circuit board. The light signal emitted by the light emitting element is transmitted to the first light receiving element through the first light-transmission portion, the channel and the second light-transmission portion to form a first light path. The second light receiving element is provided on the second surface of the circuit board in a perpendicular direction corresponding to the first light path, and is used to receive the scattered light of the impurity particles in the water flow irradiated by the light signal on the first light path, and form a second light path.

The light emitting element and the first light receiving element belong to a light transmission structure. When the washing water flows through the first light path, the output voltage difference of the light receiving element is inversely proportional to the turbidity. The light emitting element and the second light receiving element belong to a scattered light structure and share the same light emitting element with the first light receiving element. The output voltage difference of the second light receiving element is proportional to the turbidity. The special thing is that the second light receiving element for receiving the scattered light and the light emitting element are respectively located on the two opposite surfaces of the circuit board. The output voltage of the light receiving element is read by the microcontroller unit of the washing appliance, which determines whether the washing appliance uses the first light receiving element or the second light receiving element to calculate the water turbidity and washing time. By improving the accuracy of the water quality sensor, it is possible to avoid the subsequent problems caused by the washing appliances using the wrong wash cycle intensity (wasting washing water and electricity or failing to clean items).

Another object of the present invention is that in the water quality sensor, the circuit board comprises two protruding portions and a connecting portion located between the two protruding portions, so that the circuit board is in a “U” shape. The light emitting element and the first light receiving element are respectively disposed on the first surface of the circuit board at the two protruding portions. The light emitting element comprises a light signal emitting portion. The first light receiving element comprises a light signal receiving portion aimed at the light signal emitting portion of the light emitting element. The second light receiving element is disposed on the second surface of the circuit board at the connecting portion and comprises a light signal receiving portion facing toward the gap between the two protruding portions.

Still another object of the present invention is that in the water quality sensor, the sensing module further comprises an electrical connector located on the first surface of the circuit board at the connecting portion. The electrical connector comprises a plurality of conductive terminals therein. Each of the plurality of conductive terminals comprise a docking end portion disposed toward the cover, and a soldering end portion bent downward and bonded to the circuit board is located on the other side of the plurality of conductive terminals opposing the docking end portion. The cover is provided with an opening corresponding to the electrical connector.

Still another object of the present invention is that in the water quality sensor, the electrical connector of the sensing module is coupled to a microcontroller unit of a preset smart washing appliance, so that the microcontroller unit can adjust the power supply of the light emitting element and read the output voltage (PT1) of the first light receiving element and the output voltage (PT2) of the second light receiving element, and the steps for calculating the water turbidity and washing time using the above parameters are as follows: Step S1: the microcontroller unit reading the output voltage (PT1) of the first light receiving element; Step S2: the microcontroller unit determining whether the output voltage (PT1) of the first light receiving element is greater than or equal to the clean water voltage (V_PT1_CLEAN) multiplied by the parameter K, and then executing step S3 if yes, or executing step S4 if no; Step S3: the microcontroller unit using the output voltage (PT1) of the first light receiving element to calculate the water turbidity, and then executing step S1 again after washing for a predetermined time; and Step S4: the microcontroller unit using the output voltage (PT2) of the second light receiving element to calculate the water turbidity, and then executing step S1 again after washing for a predetermined time.

Still another object of the present invention is that in the water quality sensor, the clean water voltage (V_PT1_CLEAN) ranges from 3.5 to 4.5 V, and the parameter K ranges from 20 to 60%.

### BRIEF DESCRIPTION OF THE DRAWINGS

FIG. 1 is a schematic diagram of the structure of a prior art water quality sensor.

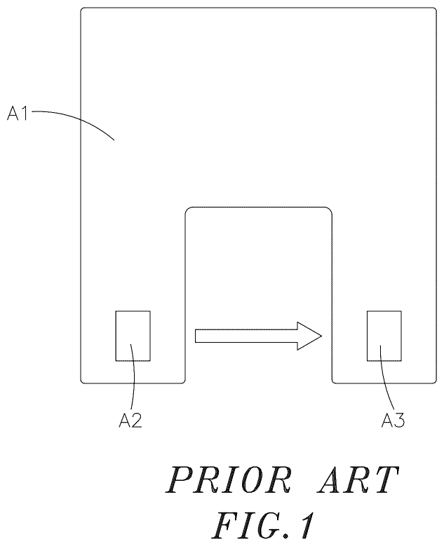

FIG. 2 is a curve graph showing the ratio of turbidity to output voltage of a conventional light receiving element.

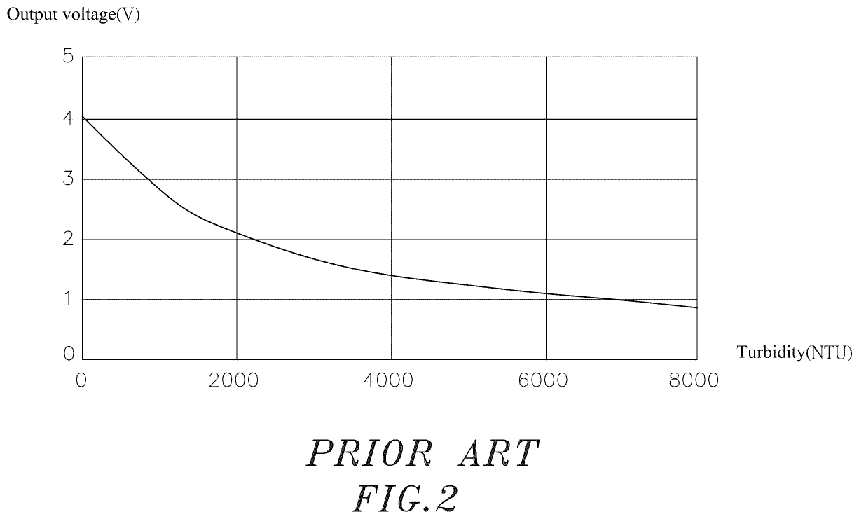

FIG. 3 is a ratio bar graph of the conventional light receiving element at different turbidities and output voltage differences.

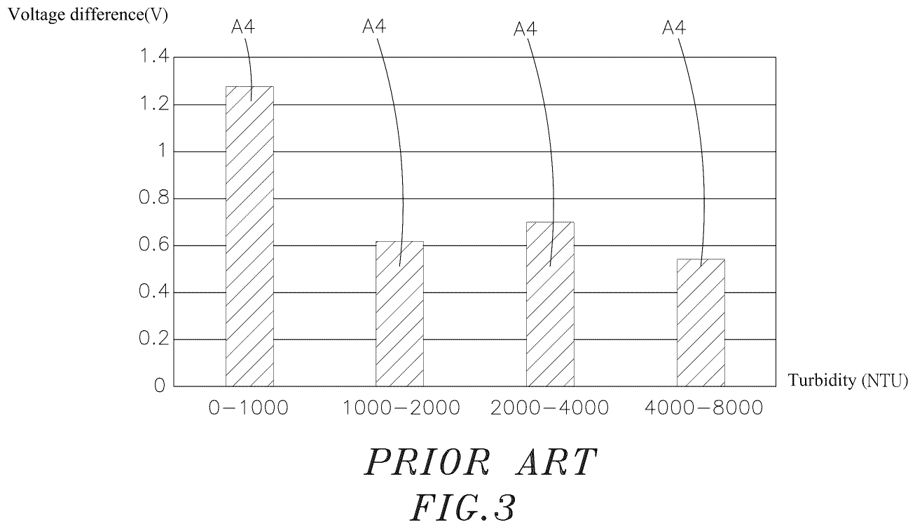

FIG. 4 is a three-dimensional external view of the water quality sensor of the present invention.

FIG. 5 is a three-dimensional external view of the water quality sensor of the present invention from another viewing angle.

FIG. 6 is a three-dimensional exploded view of the water quality sensor of the present invention.

FIG. 7 is a three-dimensional exploded view of the water quality sensor of the present invention from another viewing angle.

FIG. 8 is a three-dimensional external view of the circuit board of the present invention.

FIG. 9 is a flow chart of the method for calculating the water turbidity and washing time of the water quality sensor of the present invention.

FIG. 10 is a ratio bar graph of the present invention and the conventional light receiving element at different turbidities and output voltage differences.

FIG. 11 is another bar graph of the present invention and the conventional light receiving element at different turbidities and output voltage differences.

### DETAILED DESCRIPTION OF THE PREFERRED EMBODIMENT

In order to achieve the above-mentioned objects and effect, the technical means and structure adopted by the present invention are described in detail with reference to the preferred embodiment of the present invention, and its features and functions are as follows for a complete understanding.

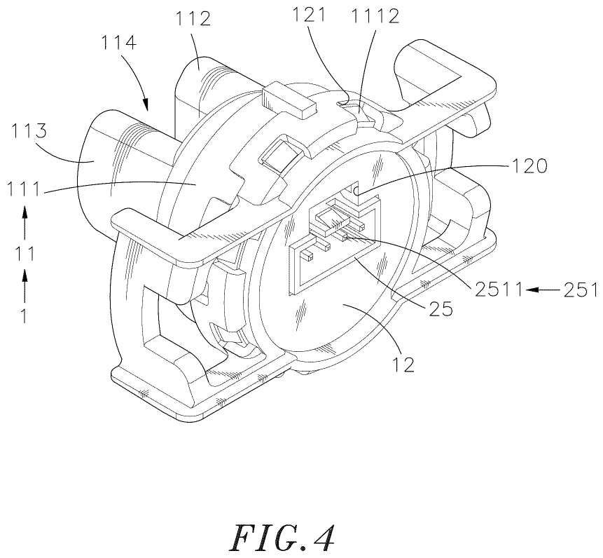

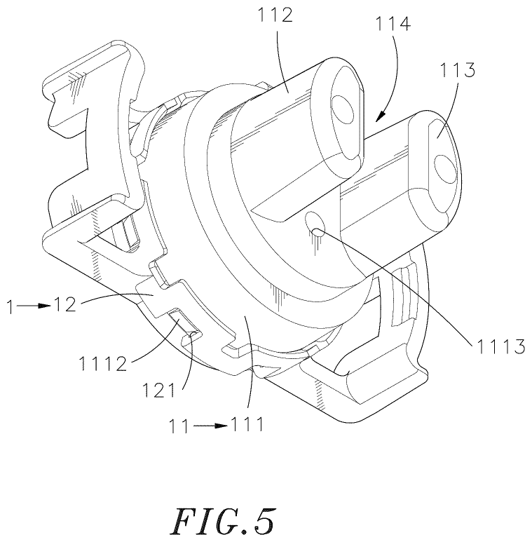

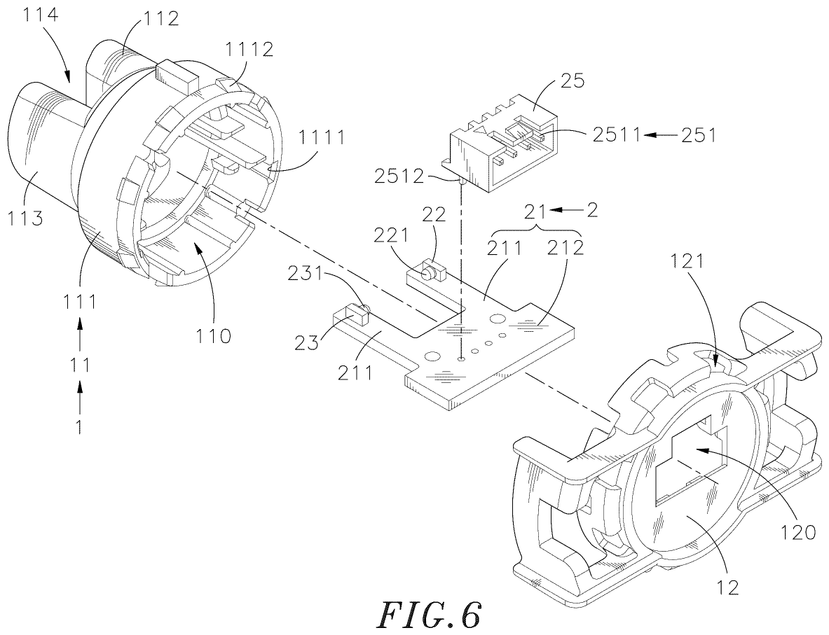

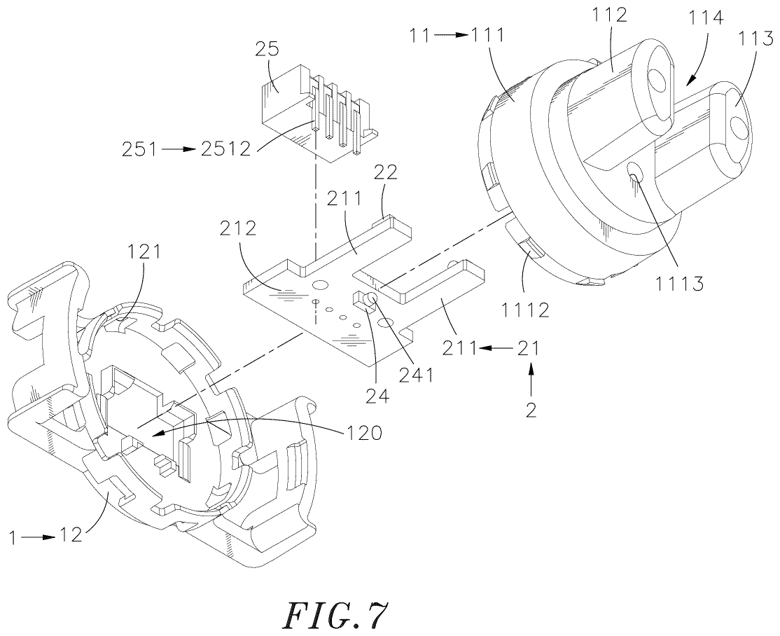

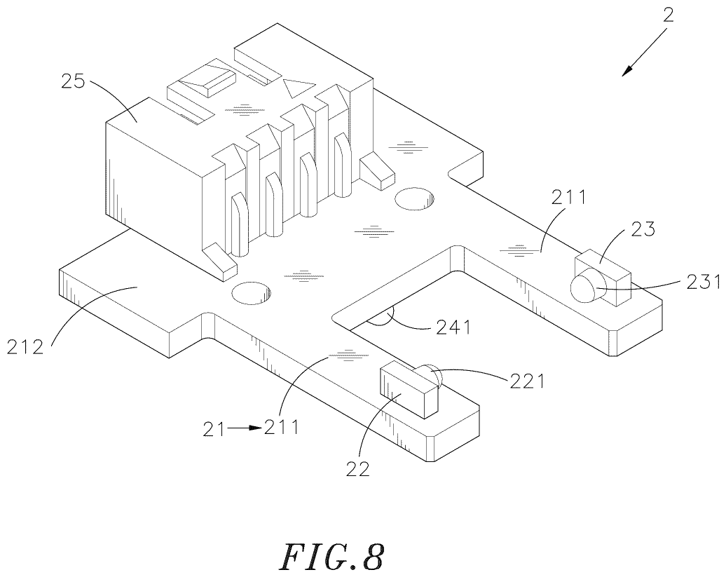

Please refer to FIGS. 4-8, which are respectively a three-dimensional external view of the water quality sensor of the present invention, a three-dimensional external view of the water quality sensor of the present invention from another viewing angle, a three-dimensional exploded view of the water quality sensor of the present invention, a three-dimensional exploded view of the water quality sensor of the present invention from another viewing angle and a three-dimensional external view of the circuit board of the present invention. As can be clearly seen from the figures, the water quality sensor of the present invention mainly comprises: a housing 1 and a sensing module 2. The principle of water quality sensing is to transmit an infrared (IR) light source into the water, and then the light in the water is received by a phototransistor and converted into electric current. This electric current can be converted into the corresponding turbidity standard according to the relationship between light power and water turbidity. Its main components and features are described as follows:

The housing 1 comprises a holder base 11 and a cover 12 which can be connected to each other. The holder base 11 has a holder base body 111, a first light-transmission portion 112 and a second light-transmission portion 113 protruded from one side of the holder base body 111, a channel 114 formed between the first light-transmission portion 112 and the second light-transmission portion 113 for water to flow through, and a receiving chamber 110 defined in the holder base body 111 in communication with the first light-transmission portion 112 and the second light-transmission portion 113.

The sensing module 2 comprises a circuit board 21 installed in the receiving chamber 110 and extended into the first light-transmission portion 112 and the second light-transmission portion 113, a light emitting element 22 provided on a first surface of the circuit board 21 and disposed in the first light-transmission portion 112, at least one first light receiving element 23 provided on the first surface of the circuit board 21 and disposed in the second light-transmission portion 113, and at least one second light receiving element 24 provided on an opposing second surface of the circuit board 21. The light signal emitted by the light emitting element 22 is transmitted to the first light receiving element 23 through the first light-transmission portion 112, the channel 114 and the second light-transmission portion 113 to form a first light path. The second light receiving element 24 is provided on the second surface of the circuit board 21 in a perpendicular direction corresponding to the first light path, and is used to receive the scattered light of the impurity particles in the water flow irradiated by the light signal on the first light path, and form a second light path.

The inner wall surface of the holder base body 111 of the holder base 11 is further provided with two positioning grooves 1111 for two side edges of the circuit board 21 to be engaged and positioned; and the outer edge of the cylindrical holder base body 111 of the holder base 11 is provided with a plurality of buckle blocks 1112 protruding outwardly. The cover 12 is provided with a plurality of buckle grooves 121 in the central cylindrical wall surface thereof. The holder base 11 and the cover 12 are combined by engaging the buckle blocks 1112 with the buckle grooves 121 respectively. The bottom surface of the holder base body 111 of the holder base 11 between the first light-transmission portion 112 and the second light-transmission portion 113 is provided with a window 1113 for the at least one second light receiving element 24 to receive the scattered light. The window 1113 is a transparent structure, a textured structure or a polished structure, and the best embodiment is a polished structure.

The above-mentioned circuit board 21 comprises two protruding portions 211 and a connecting portion 212 located between the two protruding portions 211, so that the circuit board 21 is generally in a “U” shape. The light emitting element 22 and the first light receiving element 23 are respectively disposed on the first surface of the circuit board 21 at the two protruding portions 211, and a light signal emitting portion 221 of the light emitting element 22 and a light signal receiving portion 231 of the first light receiving element 23 are aimed at each other. The second light receiving element 24 is disposed on the second surface of the circuit board 21 at the connecting portion 212 with a light signal receiving portion 241 facing toward the gap between the two protruding portions 211. The light emitting element 22 is formed of an infrared light emitting diode (IR LED), and the first light receiving element 23 and the second light receiving element 24 are both formed of a photodiode. The circuit board 21 further comprises an electrical connector 25 located on the first surface thereof at the connecting portion 212. The electrical connector 25 comprises a plurality of conductive terminals 251 therein. Each of the plurality of conductive terminals 251 comprise a docking end portion 2511 disposed toward the cover 12, and a soldering end portion 2512 bent downward and bonded to the circuit board 21 is located on the other side of the plurality of conductive terminals 251 opposing the docking end portion 2511. The cover 12 has an opening 120 corresponding to the electrical connector 25.

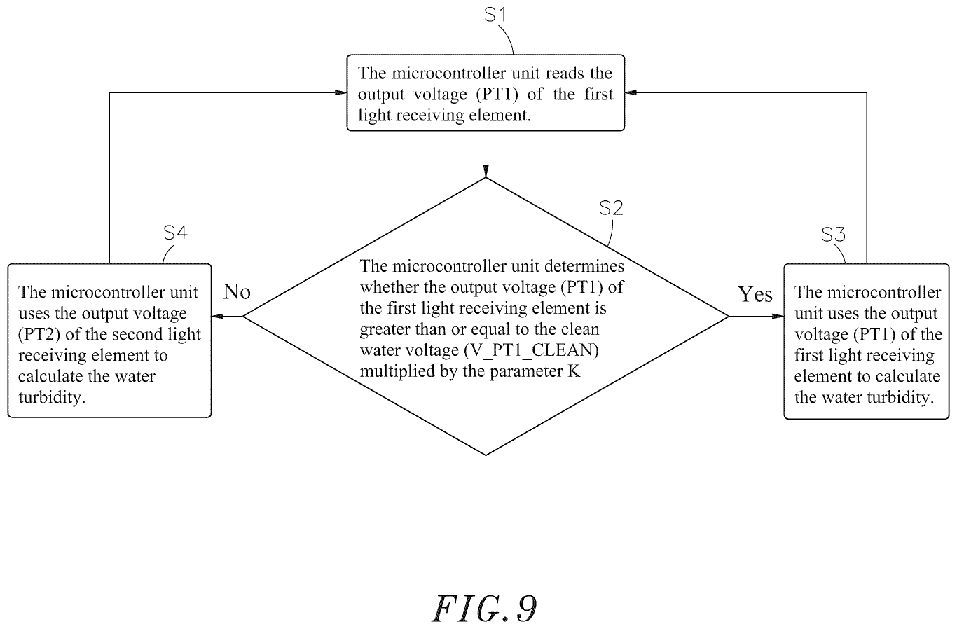

Please refer to FIG. 9, which is a flow chart of the method for calculating the water turbidity and washing time of the water quality sensor of the present invention. The electrical connector 25 of the sensing module 2 disclosed above is further coupled to a microcontroller unit (MCU, not shown) of a preset smart washing appliance, so that the MCU can adjust the power supply of the light emitting element 22 and read the output voltage (PT1) of the first light receiving element 23 and the output voltage (PT2) of the second light receiving element 24. The steps for calculating the water turbidity and washing time using the above parameters are as follows:
Step S1: The microcontroller unit reads the output voltage (PT1) of the first light receiving element 23.
Step S2: The microcontroller unit determines whether the output voltage (PT1) of the first light receiving element 23 is greater than or equal to the clean water voltage (V_PT1_CLEAN) multiplied by the parameter K. If yes, executes step S3; if no, executes step S4.
Step S3: The microcontroller unit uses the output voltage (PT1) of the first light receiving element 23 to calculate the water turbidity, and after washing for a predetermined time, executes step S1 again.
Step S4: The microcontroller unit uses the output voltage (PT2) of the second light receiving element 24 to calculate the water turbidity, and after washing for a predetermined time, executes step S1 again.

Wherein the clean water voltage (V_PT1_CLEAN) ranges from 3.5 to 4.5 V, and the parameter K ranges from 20 to 60%. The value of the parameter K is adjusted according to the type of the smart washing appliance and the water quality.

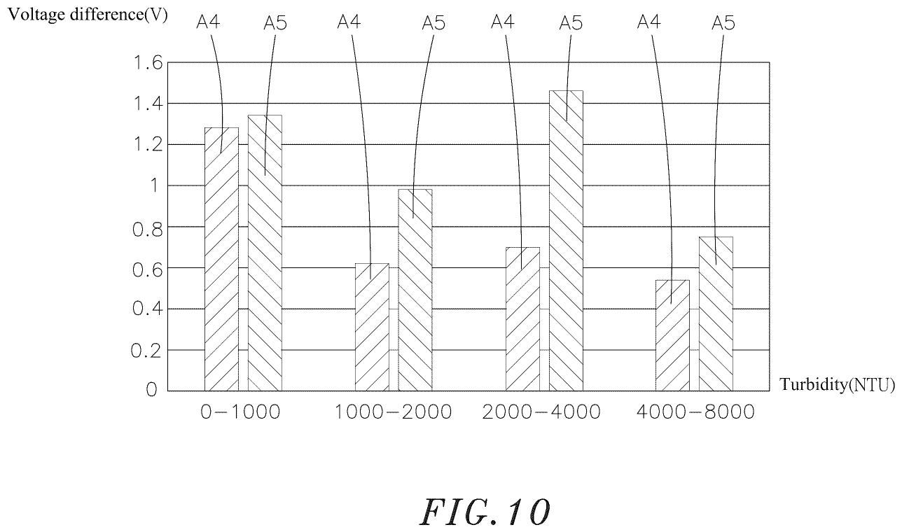

Please refer to FIG. 10, which is a ratio bar graph of the present invention and the conventional light receiving element at different turbidities and output voltage differences. When the parameter K=35% and the clean water voltage V_PT1_CLEAN is V=4V±0.2V, the effect is the best. The 0-4000 NTU (Nephelometric Turbidity Unit) algorithm is shown in the following table. The test results are as follows. The output voltage (PT1) of the first light receiving element 23 can be used to determine the turbidity of 0-1000 NTU. The remaining 1001-4000 NTU can be calculated by using the output voltage (PT2) of the second light receiving element 24.

| PT1Voltage ≥ V_PT1_CLEAN*3 | PT1Voltage < V_PT1_CLEAN*3 |
|---|---|
| 5% | 5% |
| Use PT1Voltage to determine | Use PT2Voltage to calculate |
| the turbidity in water | the turbidity in water |
| 0-1000 NTU | 1001-4000 NTU |

According to FIG. 10, the parallel bars of the turbidity/voltage difference ratios in each turbidity range are: On the left is the turbidity/voltage difference ratio of the prior art A4 (using only one light receiving element); on the right is the turbidity/voltage difference ratio of the present invention A5 (using both the first light receiving element 23 and the second light receiving elements 24). It is known that the voltage difference obtained by a single light receiving element in the turbidity range of 1000˜2000 NTU is about 0.6V, resulting in reduced resolution and easy to cause misjudgment problems. When the voltage difference obtained by two light receiving elements of the present invention in the turbidity range of 1000˜2000 NTU is very close to 1V, and the increased voltage difference is sufficient to enable the water quality sensor to form an accurate judgment, and will not cause the known problems of wasting washing water and electricity or failing to clean the items.

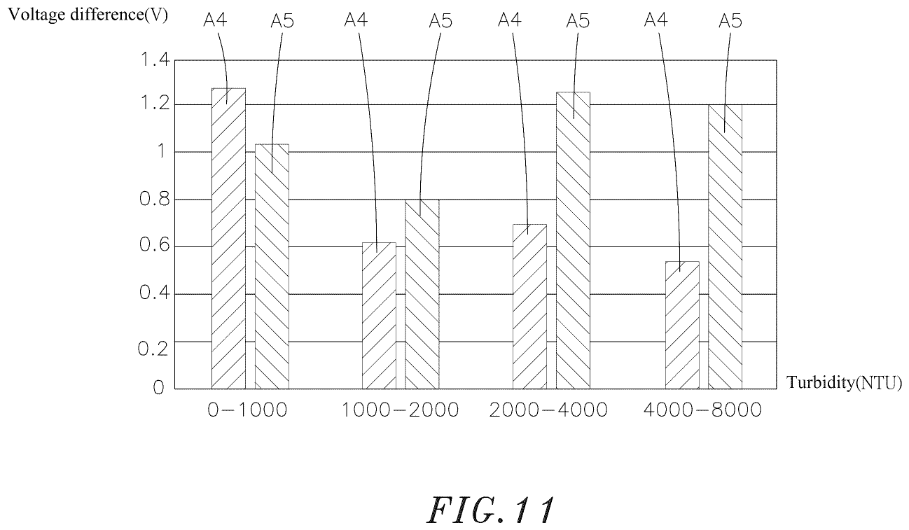

Please refer to FIG. 11, which is another bar graph of the present invention and the conventional light receiving element at different turbidities and output voltage differences. The best effect is achieved when the parameter K=35% and the clean water voltage V_PT1_CLEAN is V=4V±0.2V. The 0-8000 NTU algorithm is shown in the following table. The test results are as follows. The output voltage (PT1) of the light receiving element 23 can be used to determine the turbidity of 0-2000 NTU. The remaining turbidity of 2001-8000 NTU can be calculated by using the output voltage (PT2) of the second light receiving element 24.

| PT1Voltage ≥ V_PT1_CLEAN*3 | PT1Voltage < V_PT1_CLEAN*3 |
|---|---|
| 5% | 5% |
| Use PT1Voltage to determine | Use PT2Voltage to calculate |
| the turbidity in water | the turbidity in water |
| 0-2000 NTU | 2001-8000 NTU |

According to FIG. 11, the parallel bars of the turbidity/voltage difference ratios in each turbidity range are: On the left is the turbidity/voltage difference ratio of the prior art A4 (using only one light receiving element); on the right is the turbidity/voltage difference ratio of the present invention A5 (using both the first light receiving element 23 and the second light receiving elements 24). It is known that the voltage difference obtained by a single light receiving element in the turbidity range of 1000˜2000 NTU is about 0.6V, resulting in reduced resolution and easy to cause misjudgment problems. When the voltage difference obtained by two light receiving elements of the present invention in the turbidity range of 1000˜2000 NTU is very close to 0.8V, and the increased voltage difference is sufficient to enable the water quality sensor to form an accurate judgment, and will not cause the known problems of wasting washing water and electricity or failing to clean the items.

The main feature of the present invention is that the light emitting element 22 and the first light receiving element 23 belong to a light transmission structure. When the washing water flows through the first light path, the output voltage difference of the first light receiving element 23 is inversely proportional to the turbidity. The light emitting element 22 and the second light receiving element 24 belong to a scattered light structure and share the same light emitting element 22 with the first light receiving element 23. The output voltage difference of the second light receiving element 24 is proportional to the turbidity. The special thing is that the second light receiving element 24 for receiving the scattered light and the light emitting element 22 are respectively located on the two opposing surfaces of the circuit board 21. According to the output voltage (PT1) of the first light receiving element 23 read by the microcontroller unit of the washing appliance, which determines whether the washing appliance uses the first light receiving element 23 or the second light receiving element 24 to calculate the water turbidity and washing time. By improving the accuracy of the water quality sensor, it is possible to avoid the subsequent problems caused by the washing appliance using the wrong wash cycle intensity (wasting washing water and electricity or failing to clean items).

The above is only a preferred embodiment of the present invention, and does not limit the patent scope of the present invention. Therefore, all simple modifications and equivalent structural changes made by using the contents of the description and drawings of the present invention should be included in the patent scope of the present invention and should be declared.

In summary, the water quality sensor of the present invention can truly achieve its efficacy and purpose when used, so the present invention is truly an invention with excellent practicality. In order to meet the application requirements for invention patents, we have filed an application in accordance with the law. We hope that the review committee will approve this case as soon as possible to protect the inventor's hard work in research and development. If the review committee has any questions, please feel free to write to us for instructions. The inventor will do his best to cooperate and will be grateful for the convenience.

What the invention claimed is:

1. A water quality sensor, comprising:
a housing comprising a holder base and a cover which can be connected to each other, said holder base comprising a holder base body, a first light-transmission portion and a second light-transmission portion protruded from one side of said holder base body, a channel formed between said first light-transmission portion and said second light-transmission portion for water to flow through, and a receiving chamber defined in said holder base body in communication with said first light-transmission portion and said second light-transmission portion; and
a sensing module comprising a circuit board installed in said receiving chamber and extended into said first light-transmission portion and said second light-transmission portion, a light emitting element provided on a first surface of said circuit board and disposed in said first light-transmission portion, at least one first light receiving element provided on the first surface of said circuit board and disposed in said second light-transmission portion, and at least one second light receiving element provided on an opposing second surface of said circuit board, said light emitting element being used to transmit a light signal to said first light receiving element through said first light-transmission portion, said channel and said second light-transmission portion to form a first light path, said second light receiving element being provided on the second surface of said circuit board in a perpendicular direction corresponding to the first light path and being used to receive scattered light of impurity particles in the water flow irradiated by the light signal on the first light path and form a second light path.

2. The water quality sensor as claimed in claim 1, wherein said circuit board comprises two protruding portions and a connecting portion located between said two protruding portions, so that said circuit board is in a “U” shape; said light emitting element and said first light receiving element are respectively disposed on the first surface of said circuit board at said two protruding portions, said light emitting element comprising a light signal emitting portion, said first light receiving element comprising a light signal receiving portion aimed at said light signal emitting portion of said light emitting element, said second light receiving element is disposed on the second surface of said circuit board at said connecting portion and comprising a light signal receiving portion facing toward the gap between said two protruding portions.

3. The water quality sensor as claimed in claim 2, wherein said circuit board further comprises an electrical connector located on the first surface thereof at said connecting portion, said electrical connector comprising a plurality of conductive terminals therein, each of said plurality of conductive terminals comprising a docking end portion disposed toward said cover and a soldering end portion bent downward and bonded to said circuit board is located on the other side of said plurality of conductive terminals opposing said docking end portion; said cover is provided with an opening corresponding to said electrical connector.

4. The water quality sensor as claimed in claim 1, wherein said holder base body of said holder base is provided with two positioning grooves on an inner wall surface thereof for engaging and positioning two opposite side edges of said circuit board.

5. The water quality sensor as claimed in claim 1, wherein said holder base body of said holder base is in a cylindrical shape and is provided with a plurality of buckle blocks protruding outwardly; said cover is provided with a plurality of buckle grooves in a central cylindrical wall surface thereof; said holder base and said cover are combined by engaging said buckle blocks with said buckle grooves respectively.

6. The water quality sensor as claimed in claim 1, wherein said holder base body of said holder base is provided with a window at a bottom surface thereof between said first light-transmission portion and said second light-transmission portion for said second light receiving element to receive the scattered light, said window being selectively a transparent structure, a textured structure or a polished structure.

7. The water quality sensor as claimed in claim 1, wherein said electrical connector of said sensing module is coupled to a microcontroller unit of a preset smart washing appliance, so that the microcontroller unit is capable of adjusting the power supply of said light emitting element and reading an output voltage (PT1) of said first light receiving element and an output voltage (PT2) of said second light receiving element, and the steps for calculating the water turbidity and washing time using the above parameters are as follows:
Step S1: the microcontroller unit reading the output voltage (PT1) of said first light receiving element;
Step S2: the microcontroller unit determining whether the output voltage (PT1) of said first light receiving element is greater than or equal to the clean water voltage (V_PT1_CLEAN) multiplied by the parameter K, and then executing step S3 if yes, or executing step S4 if no;
Step S3: the microcontroller unit using the output voltage (PT1) of said first light receiving element to calculate the water turbidity, and then executing step S1 again after washing for a predetermined time; and
Step S4: the microcontroller unit using the output voltage (PT2) of said second light receiving element to calculate the water turbidity, and then executing step S1 again after washing for a predetermined time.

8. The water quality sensor as claimed in claim 7, wherein the clean water voltage (V_PT1_CLEAN) ranges from 3.5 to 4.5 V, and the parameter K ranges from 20 to 60%.

9. The water quality sensor as claimed in claim 1, wherein said first light receiving element operates in the water flow turbidity range of 0˜1000 NTU; and said second light receiving element operates in the water flow turbidity range of 1001˜4000 NTU.

10. The water quality sensor as claimed in claim 1, wherein said first light receiving element operates in the water flow turbidity range of 0˜2000 NTU; and said second light receiving element operates in the water flow turbidity range of 2001˜8000 NTU.
